(19) United States
(12) Patent Application Publication
(10) Pub. No.: US 2010/0143472 A1
(43) Pub. Date: Jun. 10, 2010

(54) OSMOTIC FORM FOR CONTROLLED RELEASE OF ACTIVE PRINCIPLES

(75) Inventor: Miller Nunes de Freitas, Sao Paulo (BR)

Correspondence Address: OBLON, SPIVAK, MCCLELLAND MAIER & NEUSTADT, L.L.P., 1940 DUKE STREET, ALEXANDRIA, VA, 22314, US

(73) Assignee: LIBBS FARMACEUTICA LTDA., Barra Funda, Sao Paulo - SP (BR)

(21) Appl. No.: 12/597,799

(22) PCT Filed: Apr. 24, 2008
(86) PCT No.: PCT/BR08/00121

§ 371 Date: Oct. 27, 2009

(30) Foreign Application Priority Data
Apr. 27, 2007 (BR) PI0701904-1

(51) Int. Cl. A61K 9/24; A61K 31/403; B05D 3/00

(52) U.S. Cl. 424/473; 514/411; 427/2.14; 424/465

(57) ABSTRACT

The present invention refers to a tablet-shaped osmotic release system providing, in a controlled way, active principles which solubility depends on the pH of the medium, simultaneously providing appropriate solubilization throughout the gastrointestinal tract. The pharmaceutical osmotic release system comprises of a pharmaceutical layer, which contains at least one active principle in a solid solution, a propelling layer, which contains at least one osmopolymer and at least one osmoagent, a semipermeable coating involving both layers, and at least one orifice in the semipermeable coating at the side of the pharmaceutical layer.

### FIELD OF THE INVENTION

The present invention refers to a new tablet-shaped release system that provides, in a controlled way, active principles, which solubility depends on the pH of the medium that simultaneously provides an appropriate solubilization throughout the whole gastrointestinal tract.

### BACKGROUND OF THE INVENTION

The efficacy of oral medicines is related to bioavailability, which may be defined as the quantity and rate at which a given active principle becomes available in the place of action. This characteristic is directly associated with the passage of the active principle from the gastrointestinal tract (GIT) to the blood flow, i.e. the absorption of the substance.

The (GIT) is made of digestive structures extending from the mouth to the anus, each one having physiological factors of its own, which may affect the bioavailability of the active principle, such as pH, components of the gastrointestinal liquid, rate of gastric emptying, viscosity, absorption surface, nature of the biological membrane, etc.

For the absorption of an active principle, the last must be fully solubilized in the place of action (GIT). For pharmaceutical products presenting solubility depending on the low pH of the medium, the oral pharmaceutical forms should provide the active principle in the stomach, which has acid pH, to enable solubilization and, consequently, appropriate absorption.

It is known that the faster the absorption in the (GIT) is, lower will be the influence of physiological factors in the process. On the other hand, said availability or selective solubilization causes quick and high concentration of the active principle in the plasma, responsible for immediate therapeutic response.

For certain substances, the absorption peaks and the plasmatic fluctuations caused by quick initial absorption are responsible for undesirable side effects, such as acute hypotension, headaches, nausea, among other.

In general, such side effects are reduced by the sustained release oral formulations. These formulations do not allow quick initial absorption, by providing constant dosage of active principle for a given period of time.

However, this alternative known by the skilled person in the art is not feasible when the active principle has solubility depending on low pH, since as the pharmaceutical form is displaced by the (GIT), constituted by structures with different characteristics, the pH increases and, consequently, the dissolution and absorption of the active principle become lower. The negative impact in absorption makes the development of pharmaceutical forms providing sustained release throughout the (GIT), become difficult.

A few alternatives to improve release or absorption have been developed. The international publication WO 05/041929, for example, proposes a release system combining, in one same formulation, the desired active principle, a solubilizer (substance with surfactant properties) and a release modulator to sustain the release.

The publication WO 99/24017 discloses a matrix containing an active principle with low solubility for oral administration. The disclosed systems include: (a) tablet matrix containing hydroxypropylmethylcellulose and carbomer as excipients to control release rate; (b) immediate release core, covered with an enteric polymer or for sustained release; and (c) spheres covered with glycerylmonostearate and glyceryldistearate.

The publication WO 02/092078 also describes a composition using specific excipients for controlled release. Other technologies aiming to increase circulation or dissolution time of these active principles include miniaturized release systems, known as micropump or hydrophilic matrices. For example, the publication by Tenero et al (Am. J. Cardiol. 2006; 98 (suppl.): 5L-16L) discloses complex formulations containing three microparticulate components covered with pH-sensitive polymers, making the release depend on the pH of said polymer.

The publication WO 01/74357 presents formulations providing for improvements in the absorption of said active principles in the lower parts of the (GIT) by increasing contact surface.

However, said technologies present unsatisfactory results concerning simultaneous bioavailability and solubility of the active principle and, in some cases, under high manufacturing cost. Therefore, there is the need for pharmaceutical forms with appropriate cost providing gradual release of active principles which solubility depends on low pH of the medium, thus avoiding side effects of the quick absorption into the stomach, but at the same time helping its dissolution in different place of actions while the pharmaceutical form is displaced through the gastrointestinal tract (stomach, duodenum and gut).

Also, these forms would be desired to improve patients' adhesion to the treatment, since the longer the time of permanence of the active principle in blood circulation is, the lower will be the quantity of medicine administrations.

Aiming to overcome the drawbacks mentioned above, the applicant developed a new controlled release pharmaceutical form, appropriate for active principles which solubility depends on the pH of the medium, using osmotic release technology, also known as OROS—“Oral Release Osmotic System.”

The incorporation of active principles with limited solubility in a specific push-pull type osmotic platform was made with the purpose to extend release time and consequently its plasmatic circulation. The push-pull osmotic system has been proposed as an alternative to the elementary monolithic (monolayer) system. The monolithic system comprises an osmotic core containing the pharmaceutical, covered by a semipermeable membrane and a release orifice. In operation, the osmotic core receives water from the medium around it through the semipermeable membrane, giving origin to a pharmaceutical solution which is released from the system by the orifice. Therefore, the elementary osmotic system had as a pre-requisite the dissolution of the hydrophilic pharmaceutical inside the core for later release.

With the development of the push-pull osmotic system, it has also become possible to release pharmaceuticals with hydrophobic characteristics. The push-pull system consists of a bilayer tablet wherein the first layer includes the pharmaceutical (pharmaceutical or active principle compartment) and the second layer works as a propelling layer (propelling compartment). The pharmaceutical layer is composed by a diluent and by low molecular weight polymers and the propelling layer is composed by high molecular weight osmopolymers and, eventually, by an osmoagent. The difference between an osmopolymer and an osmoagent lies on the fact that osmoagents are only responsible for establishing an osmotic pressure gradient, giving origin to a hydroactive layer. On the other hand, osmopolymers have the ability to swell in water or biological fluids, retaining a significant portion of fluid within their structure. Furthermore, osmopolymers expand at very high rates, usually showing volume increase of 2 to 50 times.

When the push-pull system makes contact with the water medium, the layers absorb water and the lower compartment, which does not have an orifice, swells and pushes the upper layer. Consequently, the upper layer becomes contracted, releasing the pharmaceutical through the orifice at constant rate and only depending on the osmotic pressure.

The new pharmaceutical form of the present invention not only provides the controlled release of the active principle, but simultaneously provides appropriate solubilization throughout the gastrointestinal tract (GIT), no matter acidity characteristics in the place of action. Among the advantages of the osmotic system of the present invention, are also included the easy characterization, quality control of the final product and lower production cost in comparison with other available skills.

### DESCRIPTION OF FIGURES

FIG. 1 shows the dissolution curve of the non-covered tablets of the present invention containing only the pharmaceutical compartment formulation.

FIG. 2 shows the active principle dissolution profile in HCl 0.1N medium from osmotic systems of the present invention covered with 8 and 10% weight gain. The values as presented at each point represent the percentage of active principle released through the time.

FIG. 3 shows the plasmatic profile of six different hydrophilic matrices submitted to a comparative pharmacokinetic assay.

FIG. 4 shows the plasmatic profile of OROS formulations of the present invention and immediate release (reference), submitted to the pharmacokinetic assay.

### DESCRIPTION OF THE INVENTION

The pharmaceutical form of controlled release of the present invention comprises:

(a) a pharmaceutical layer, containing at least one active principle which solubility depends on the low pH of the medium, in solid solution;

(b) a propelling layer containing at least one osmopolymer with high molecular weight and eventually at least one osmoagent;

(c) at least one semipermeable coating involving both layers; and

(d) at least one orifice at the pharmaceutical layer to release the active principle.

By “active principle which solubility depends on the low pH of the medium” is understood the active substance with low hydrosolubility (approximately 0.001 mg/mL) when released in a site of action having pH higher than 7. Particularly, these substances may be weakly basic with pKa of about 7.5, and they present considerable hydrosolubility with the formation of the corresponding ionized forms thereof.

Based on solubility and permeability characteristics, a few active principles are classified according to the Biopharmaceutical Classification System, as used by various regulating agencies: ANVISA (Health Surveillance National Agency—Agenda Nacional de Vigilância Sanitaria, Brazil), FDA (Food and Drug Administration, U.S.A.) and EMEA (European Agency for the Evaluation of Medicinal Products, Europe). Class I includes active principles of high solubility and high permeability; class II includes the ones of low solubility and high permeability; class III includes the ones of high solubility and low permeability and class IV includes the ones of low solubility and low permeability.

Compounds with low solubility of the present invention are included in classes II and IV of the Biopharmaceutical Composition System. Particularly, the active principles of the present invention are selected from one or more among: amiodarone, atazanavir, atorvastatin, azithromycin, benazepril, bicalutamide, candesartan cilexetil, carbamazepin, carisoprodol, carvedilol, celecoxib, clarithromycin, diazepam, divalproex, docetaxel, donepezil, efavirenz, etodolac, ezetimibe, phenofibrate, finasterid, gemfibrozil, glimepiride, gliburide, ibuprofene, indapamide, indometacin, irbesartan, cetoconazol, lansoprazol, loratadin, lovastatin, meclizin, metaxalone, moxifloxacin, mycophenolate mofetil, nabumetone, nelfinavir, olmesartan medoxomil, pioglitazone, prednisone, raloxifene, risperidone, ritonavir, rofecoxibe, sinvastatin, spironolactone, drospirenone, tachrolimus, temazepam, valdecoxibe, valsartan, ziprasidone, isomers, salts, solvates, hydrates, polymorphs or the derivatives thereof. More particularly, the principle of the present invention is carvedilol.

The amounts of active principle in pharmaceutical forms of the present invention may be in the range of about 3 to about 80 mg, particularly in the range of about 25 to about 50 mg per dosage unit.

Any other active principles may be included in the pharmaceutical form of the present invention. In all cases, bioavailability and solubility characteristics are advantageously improved.

The osmopolymers of the present invention are selected from high molecular weight polyoxyethylene oxides or the derivatives thereof. The osmoagents of the present invention are selected from soluble salts of inorganic acids, such as magnesium chloride or sulphate, lithium, sodium or potassium chloride; soluble salts of organic acids, such as sodium or potassium acetate, magnesium succinate, sodium benzoate, sodium citrate, sodium ascorbate; carbohydrates, such as arabinose, ribose, xylose, glucose, fructose, galactose, mannose, sucrose, maltose, lactose, raffinose; hydrosoluble aminoacids, such as glycine, leukine, alanine, methionine; organic polymeric osmoagents, such as sodium carboxymethylcellulose, hydroxypropylmethylcellulose, hydroxyethylmethylcellulose, polyvinylpirrolidone, polyoxyethylene oxide, carbomers and polyacrylamides.

The semipermeable coating may be selected from one or more polymers derivated from cellulose, such as cellulose diacetate, cellulose triacetate, cellulose propionate, cellulose acetate butirate, cellulose esters such as ethylcellulose, more particularly cellulose acetate and esters of acrylic and methacrylic acid.

Advantageously, the coating should contain a plastifying substance in its formulation since it makes the coating polymer becomes more flexible and less friable, being easier to cover various shapes of tablets. The plastifiers may be one or more from polyethylene glycol, diatecetin, diethyl tartarate, triacetin, triethyl citrate, dibutyl sebacate, more particularly polyethylene glycol.

A particular coating comprises 3.5% of cellulose acetate, 0.5% of polyethylene glycol (commercially available as Macrogol 3350), 86.5% of dichloromethane or acetone and 9.5% of ethanol or water.

For the membrane orifice, laser perforation may be used due to its high precision and agility of the process as offered by the equipment. Furthermore, for the release orifice to allow appropriate performance of the release system, it should have diameter from about 0.15 mm to about 2.0 mm, more particularly from about 0.25 mm to about 1.41 mm.

The precision of the used orifice was reached with equipment parameters of 100% precision, point magnitude of 0.495 mm, point height and width of 0.3 mm×0.3 mm, respectively, static mode, working time of 300 microseconds and laser distance of 41 mm.

The pharmaceutical forms of the present invention, besides providing for the active principle release for about 24 hours, also help their absorption, characteristics that result in the reduction of side effects and, consequently, in a better treatment efficacy.

Such enhanced absorption throughout the gastrointestinal tract comes from the use of a solid solution in the pharmaceutical layer, comprising:

(i) at least one active principle which solubility depends on the low pH of the medium;

(ii) at least one hydrophilic adjuvant;

(iii) at least a lower alcohol; and

(iv) optionally at least one lubricant.

The hydrophilic adjuvant is selected from one or more among polyoxyethylene stearate, copolymer of polyoxyethylene-polyoxypropylene, sugars of hydrogenated isomaltulose type, hydroxypropylmethylcellulose, polyvinylpirrolidone and polyethylene glycol with molecular weights in the range of about 1,000 to about 20,000, more particularly polyethylene glycol with molecular weight of about 6,000.

The lower alcohol is selected from one or more C₁ to C₅ alcohols or the derivatives thereof, being particularly ethanol. The lubricant may be selected from one or more among magnesium stearate, stearic acid, sodium stearyl fumarate, more particularly being magnesium stearate.

In another aspect, the present invention refers to the preparation process of the pharmaceutical form of controlled release, consisting of:

(a) preparation of the pharmaceutical and propelling layers;

(b) double layer compression;

(c) application of the semipermeable coating; and

(d) laser perforation.

More particularly, the present invention refers to the process to prepare a solid solution used to prepare the pharmaceutical layer, comprising the steps of:

(1) Heating at least one hydrophilic adjuvant until melt, particularly in the range of about 70 to about 80° C.;

(2) Adding at least one active principle which solubility depends on the low pH of the medium under shaking;

(3) Adding at least one lower alcohol under shaking to reduce viscosity, until the full dissolution of (2) into (1);

(4) When required, after reducing the process temperature in about 25%, adding other pharmaceutically appropriate excipients, as well as other hydrophilic adjuvants;

(5) Granulating;

(6) Drying until about 1 to about 2.5% of humidity; and

(7) Optionally, adding the lubricant.

The quantity proportion from (1) to (2) is in the range of about 1:5 to about 5:1, being particularly 2:1.

Preferably, drying is conducted in an oven or fluidized bed under temperature from about 25 to 50° C. for about five to ten hours. More particularly, the temperature should be of about 35° C. for about eight hours.

The addition of lower alcohol consists in an essential step to reduce the viscosity of the mixture and facilitate granulation, as well as to help the destruction of the active principle crystal, contributing to its solubilization.

In another aspect, the present invention also refers to the method of therapeutic treatment comprising the administration to a patient in need of the pharmaceutical form of the present invention once a day during the appropriate treatment period, as well as the use of the pharmaceutical form of the present invention once a day.

### EXAMPLES

Examples below are intended to illustrate aspects of the present invention, and do not have limitative purpose. For easy exposition, the examples presented below only refer to an active principle which solubility depends on the pH of the medium (carvedilol), however, it does not represent any limitation to the scope of the invention.

### Example 1

Process to Prepare the Active Principle Layer

500 grams of polyethylene glycol 6000 were weighted with the help of a magnetic plate, the polymer was heated to 65° C. After the melt of polyethylene glycol, the slow addition of 250 grams of carvedilol was started under mechanical shaking. Subs equently, 100 mililiters of hydrated ethanol (96%) were added and shaking was maintained until its full dissolution.

In parallel, other hydrophilic adjuvants—polyoxyethylenes (commercially available as Poliox N80 and N10) and methylcellulose (commercially available as Methocel K4000)—were mixed with the help of a High Shear granulator (from the company Silverson) with rate of 400 rpm and cutter at 120 rpm for approximately five minutes.

The carvedilol solution as prepared was taken from the magnetic plate and it was waited until the temperature reached 50° C. before starting to add of other components. After cooling, the ethanol solution of polyethylene glycol and carvedilol was added over the other components, keeping the mixture rate in the range as specified above and the rate of addition of 10 rpm. Upon reaching the point of granulation, the process was interrupted and the granulation was classified in a rotating mill by using 5 mm mesh, before being taken to the oven (35° C.), where it remained for approximately eight hours until humidity reached the 1 to 2.5% level. After the drying process, the powder was again classified in a rotating mill, but using grater mesh wish 1 mm opening.

Finally, after classifying the granulate, magnesium stearate lubricant was added and mixed for three minutes. After the step to obtain granulated powder, the compression process for finishing was started.

### Example 2

### Dissolution Assay of the Active Principle Layer

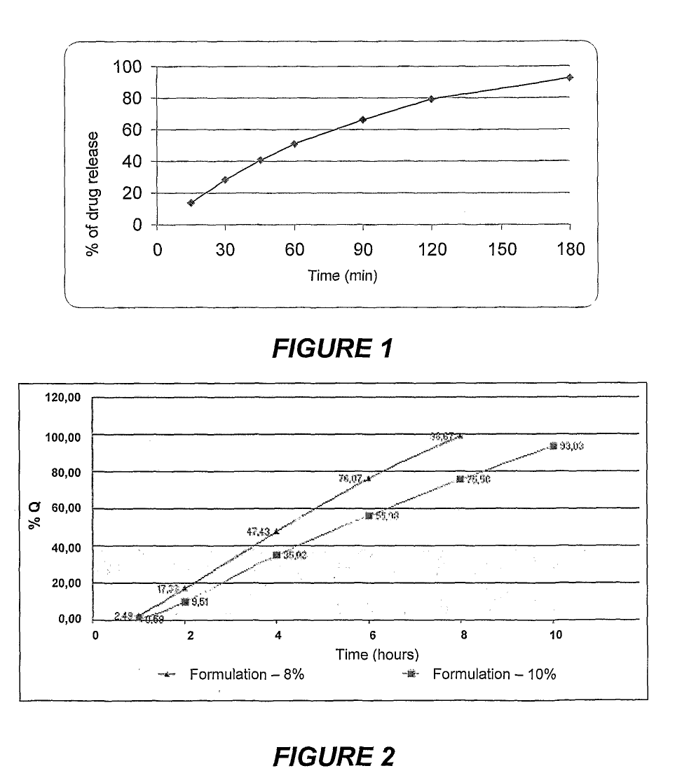

The cores produced in the Example 1 have been submitted to a dissolution assay and the result is described on FIG. 1. By analyzing the carvedilol dissolution profile from the tablets produced with the components of the active principle compartment, it is possible to observe good approximation of the kinetics of order zero as desired, besides prolonging release for a three-hour period.

### Example 3

### Process to Prepare the Pharmaceutical Form of the Invention by Double Compression

The preparation of the propelling layer was made according to the Example 1. To prepare the propelling compartment, the same procedure and equipment was used, but at lower rates.

The formulation of the propelling compartment was prepared with 1200 grams of polyoxyethylenes, 90 grams of methylcellulose, 700 grams of sodium chloride, 10 grams of magnesium stearate, 5 grams of red iron oxide and 600 mililiters of ethanol.

In the double compression process, the powder related to the pharmaceutical layer was firstly added and low compression force was used, enough to take off the core and calibrate the weight to 200 mg. The low compression force of the first compartment (pharmaceutical compartment) is required for the occurrence of the adhesion of the second compartment (propelling compartment) after the final compression or second compression. The propelling compartment or second compartment was added and its mass was calibrated to 150 mg, thus the tablet has a total average weight of 350 mg with average diameter of 10 mm, average thickness of 4.8 mm, average thickness of 4.8% and average hardness of 6.71 (specified range, but not limiting to 5-8 kgf).

After the conclusion of the core production step, a coating was made with an automated coater with semiperforated bucket, Lab Coater (Vector Corporation). The coating used comprises 3.5% of cellulose acetate, 0.5 grams of polyethylene glycol (Macrogol 3350), 86.5% of dichloromethane and 9.5% of ethanol.

Finally, laser perforation was made with equipment parameters of 100% of precision, point magnitude of 0.495 mm, static mode, working time of 300 seconds, point height and width of 0.3 mm and laser distance in relation to the product of 41 mm.

### Example 4

### Profile of In Vitro Release of the Pharmaceutical Form of Example 3

In vitro release profiles of the active principle from the osmotic system as disclosed by Example 3 and covered with 8 and 10% weight gain are presented on FIG. 2.

### Example 5

### Pharmacokinetic Assay

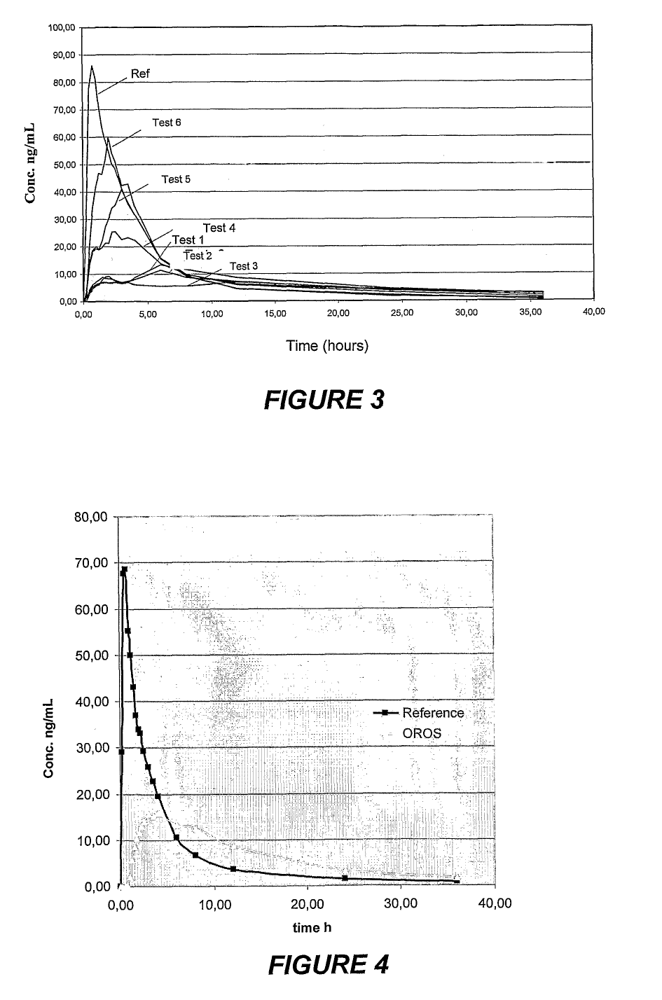

The pharmaceutical form of Example 3, as well as bioadhesive hydrophilic matrices were submitted to a pharmacokinetic pilot assay and graphs related to the pharmaceutical bioavailability after release by different formulations may be observed on FIGS. 3 and 4.

The formulations related to hydrophilic matrices as developed also with the object to reach one single daily dosage for the same active principle which solubility depends on the low pH of the medium (carvedilol) were prepared in the form of controlled release tablets. Six different formulations were prepared by varying quantity and type of gelling polymer, and submitted to a pharmacokinetic study by using healthy volunteers and collecting blood samples within pre-determined times to dose the quantity of present active principle. An immediate release formulation (reference—REF) under the same dosage was also used in the assay to serve as a comparison standard. Prolongation of the pharmaceutical elimination stage for test formulations is expected, as well as the areas under the curve (AUC) between them and the reference medicine to be similar. The graph representing plasma profiles obtained for different formulations referred to as “test” may be observed on FIG. 3.

From the results, we can observe that tests 1 and 2 showed reduced bioavailability, i.e. just a small quantity of the pharmaceutical reached the blood flow. Other test formulations promoted quicker release, reaching better bioavailability. Anyway, was observed that a satisfactory result was reached both to reduce the maximum concentration (Cₘₐₓ) as obtained and to prolong the arrival time at the plasmatic peak. However, it was not possible to increase the plasmatic level at the elimination stage. On the other hand, the quantity absorbed, as measured by the area under the curve, was reduced proportionally to the reduction of release rate.

The same pharmacokinetic assay was proposed for the pharmaceutical form of controlled release of the present invention and results are presented on FIG. 4. Results related to AUC, Cmax and the half life time in plasma (T1/2) are disclosed in the table below:

TABLE 1
COMPARATIVE RESULTS
PHARMACEUTICAL FORM OF THE INVENTION AND
IMMEDIATE RELEASE REFERENCE TABLET

| Reference | | Example 3 | |
|---|---|---|---|
| AUC | 254.63 | AUC | 236.27 |
| Cmax | 68.60 ng/mL | Cmax | 15.91 ng/mL |
| T½ | 5.40 hours | T½ | 12.17 hours |

By analyzing the obtained results, it is possible to notice that the pharmaceutical form of the present invention was able to prolong the permanence of the active principle in the blood flow (T1/2 of 12.17 hours), mainly during the elimination step, presenting plasma peak 4.3 times lower and furthermore the area under the curve is very close to the value as presented by the reference medicine (93%), indicating the possibility of two administrations substitution (immediate release) by one administration (controlled release).

Even releasing the active principle in a controlled way throughout the gastrointestinal tract and not only at the higher part wherein pH is lower, it was possible to overcome deficiencies of the alternatives in the state of the art, i.e. Cmax reduction with increase in the time of permanence of the active principle in the plasmatic flow mainly during the elimination/metabolization step. Besides reducing daily administrations and the plasmatic fluctuations, other advantages are characteristic of the system as developed:

Ability to reach a kinetic release model of zero order type (as observed in FIG. 2);

Independent release rate of gastric pH and of hydrodynamics of the medium;

Predicable release rate;

Release rates considered as high, in comparison with conventional pharmaceutical forms, controlled by difusion;

Possibility to formulate different pharmaceuticals with different water solubility ranges; and

Ability to release even a combination of pharmaceuticals.

1. A pharmaceutical form for controlled release of one active principle, wherein a solubility of the pharmaceutical form depends on a low ph of a medium, the pharmaceutical form comprising:
(a) a pharmaceutical layer comprising caverdilol c in a solid solution;
(b) a propelling layer comprising at least one osmopolymer with a high molecular weight and optionally at least one osmoagent;
(c) at least one semipermeable coating on the pharmaceutical layer and the propelling layer; and
(d) at least one orifice at the semipermeable coating at a side of the pharmaceutical layer to release the active principle.

2. The pharmaceutical form according to claim 1, wherein a quantity of active principle is in the range of 3 to 80 mg per pharmaceutical form.

3. The pharmaceutical form according to claim 2, wherein the quantity of active principle is in the range of 25 to 50 mg per pharmaceutical form.

4. The pharmaceutical form according to claim 1, wherein the at least one osmopolymer is polyoxyethylene oxide of high molecular weight.

5. The pharmaceutical form according to claim 1, wherein the at least one osmoagent is selected from the group consisting of magnesium chloride or sulphate, lithium, sodium or potassium chloride; sodium or potassium acetate, magnesium succinate, sodium benzoate, sodium citrate, sodium ascorbate; arabinose, ribose, xylose, glucose, fructose, galactose, mannose, sucrose, maltose, lactose, raffinose; alpha amino acids, such as glycine, leukine, alanine, methionine; sodium carboxymethylcellulose, hydroxypropylmethylcellulose, hydroxyethylmethylcellulose, polyvinylpyrrolidone, polyoxyethylene oxide, carbomers and polyacrylamides.

6. The pharmaceutical form according to claim 1, wherein the semipermeable coating is selected from the group consisting of cellulose acetate, cellulose diacetate, cellulose triacetate, cellulose propionate, cellulose acetate butyrate, ethyl cellulose and esters of acrylic and methacrylic acid.

7. The pharmaceutical form according to claim 1, wherein the semipermeable coating comprises at least one of polyethylene glycol, diacetin, diethyl tartarate, triacetin, triethyl citrate and dibutyl sebacate.

8. The pharmaceutical form according to claim 1, wherein the semipermeable coating comprises 3.5% of cellulose acetate, and 0.5% of polyethylene glycol.

9. The pharmaceutical form according to claim 1, wherein the orifice has a diameter of 0.15 mm to 2.0 mm.

10. The pharmaceutical form according to claim 1, wherein the orifice has a diameter of 0.25 mm to 1.41 mm.

11. The pharmaceutical form, according to claim 1, wherein the pharmaceutical layer comprises a solid solution comprising:
(i) caverdilol;
(ii) at least one hydrophilic adjuvant; and
(iii) optionally, at least one lubricant.

12. The pharmaceutical form according to claim 1, wherein the hydrophilic adjuvant is selected from the group consisting of polyoxyethylene stearate, polyoxyethylene-polyoxypropylene copolymer, sugars of hydrogenated isomaltulose type, hydroxypropylmethylcellulose, polyvinylpirrolidone and polyethylene glycol with molecular weight in the range of 1,000 to 20,000.

13. The pharmaceutical form, according to claim 12, wherein the hydrophilic adjuvant is polyethylene glycol with molecular weight of about 6,000.

14. The pharmaceutical form according to claim 11, wherein the lubricant is selected from the group consisting of magnesium stearate, stearic acid and sodium stearyl fumarate.

15. A process for preparing a pharmaceutical form according to claim 1, comprising
(a) preparing the pharmaceutical and propelling layers,
wherein a solid solution is prepared by:
(1) heating at least one hydrophilic adjuvant until 70° C. and 80° C.;
(2) adding caverdilol under shaking;
(3) adding at least one lower alcohol selected from one or more C₁ to C₅ alcohols under shaking until the full dissolution of (2) into (1);
(4) optionally, after reducing the process temperature in about 25%, adding other pharmaceutically appropriate excipients, as well as other hydrophilic adjuvants;
(5) granulating;
(6) drying until about 1 to 2.5% of humidity; and
(7) optionally, adding a lubricant;
(b) carrying out double layer compression;
(c) applying the semipermeable coating; and
(d) carrying out laser perforation.

16. The process according to claim 15, wherein the lower alcohol is ethanol.

17. The process according to claim 15, wherein the equipment parameters for the laser perforation are of 100% precision, 0.495 mm point magnitude, point height and width of 0.3 mm×0.3 mm, respectively, static mode, working time of 300 microseconds and laser distance of 41 mm.

18. The process according to claim 15, wherein a quantity ratio of (1) to (2) is in the range of 1:5 to 5:1.

19. The process according to claim 18, wherein the quantity ratio of (1) to (2) is 2:1.

20. The process according to claim 15, wherein the drying is carried out under temperature in the range of 25° C. to 50° C. for 5 to 10 hours.

21. (canceled)
